(19) United States
(12) Patent Application Publication
(10) Pub. No.: US 2025/0127587 A1
(43) Pub. Date: Apr. 24, 2025

(54) SURGICAL OPERATING SYSTEM, SURGICAL TOOL AND METHOD FOR SAFEGUARDING A SURGICAL OPERATION

(71) Applicant: CARL ZEISS MEDITEC AG, Jena (DE)

(72) Inventors: Carolin KLUSMANN, Oberkochen (DE); Tobias HARTER, Oberkochen (DE); Niklas BORCHERS, Oberkochen (DE); Matthias HILLENBRAND, Oberkochen (DE); Caglar ATAMAN, Oberkochen (DE); Florian LUX, Oberkochen (DE)

(73) Assignee: CARL ZEISS MEDITEC AG, Jena (DE)

(21) Appl. No.: 18/919,208

(22) Filed: Oct. 17, 2024

(30) Foreign Application Priority Data
Oct. 19, 2023 (DE) 10 2023 210 298.6

(51) Int. Cl. A61B 90/00; A61F 9/007; G01B 9/02091; G01B 11/14

(52) U.S. Cl. CPC A61B 90/06; A61F 9/007; G01B 9/02091; G01B 11/14; A61B 2090/061; A61F 2009/00851

(57) ABSTRACT

Provided is a surgical operating system, comprising a surgical tool, an optical sensor arranged on or in the surgical tool and configured to measure a distance (8-x) of the surgical tool from a tissue for a plurality of directions (6-x) and/or for a plurality of mutually separated punctiform spatial regions (7-x), an optical coherence tomography measuring device connected to the optical sensor and configured to determine the respective distance (8-x) from the tissue for the plurality of directions (6-x) and/or punctiform spatial regions (7-x) by means of the sensor and to provide the determined distances (8-x). Also provided are a surgical tool and a method for safeguarding a surgical operation.

The invention relates to a surgical operating system, a surgical tool and a method for safeguarding a surgical operation.

In surgical operations, especially in the field of microsurgery, the intention is to manipulate tissue in one region, for example by virtue of the tissue being removed, whereas tissue in adjacent regions should be protected. In eye surgery, for example, it is therefore necessary to estimate the distance of a surgical tool introduced into the eye from a tissue layer to be examined or treated, for example the retina in the eye. For example, in a vitrectomy, it is known practice to perform such a distance estimation with the aid of a shadow that is caused by the surgical instrument. To this end, a surgical microscope is used for imaging and magnification purposes, and a shadow cast on the retina by a light pipette introduced through a trocar is visually evaluated by a surgeon. However, this procedure is inaccurate, requires much experience and is ultimately also linked to a risk of injuring healthy tissue of the eye during the operation.

It is therefore desirable to improve such a distance estimation.

US 2011/0 106 102 λ1 has disclosed a surgical instrument. The surgical instrument comprises a surgical tool having a proximal end and a distal end, and also an optical sensor, at least one part of which is fastened to the surgical tool. The surgical tool has a section suitable for providing a reference section of the surgical tool, and the optical sensor has an end that is fastened relative to the reference section of the surgical tool such that the reference section of the surgical tool is captured together with tissue that is in the vicinity of the distal end of the surgical instrument, or in contact with the latter, during use.

A. Abid et al., A Smart Vitrector Equipped by a Fiber-Based OCT Sensor Mitigates Intentional Attempts at Creating Iatrogenic Retinal Breaks During Vitrectomy in Pigs, Translational Vision Science & Technology 10(13): 19, November 2021, DOI: 10.1167/tvst.10.13.19, has disclosed an OCT-based optical sensor for determining distances on a surgical tool.

The problem addressed by the invention is that of improving a surgical operating system, a surgical tool and a method for safeguarding a surgical operation, in particular in view of determining a distance from a tissue.

According to the invention, this problem is solved by a surgical operating system having the features of claim 1, a surgical tool having the features of claim 26 and a method having the features of claim 28. Advantageous configurations of the invention are evident from the dependent claims.

One of the fundamental concepts of the invention is that of arranging an optical sensor on or in the surgical tool, wherein the optical sensor is configured to measure a distance of the surgical tool, in particular of a distal end thereof, from a tissue for a plurality of directions and/or for a plurality of mutually separated punctiform spatial regions. As a result, a distance from tissue, for example the retina, can be measured in a plurality of directions around the surgical tool, in particular around the distal end, during an operation, for example on or in an eye. To this end, the optical sensor is connected to an optical coherence tomography (OCT) measuring device. This optical coherence tomography measuring device is configured to determine the respective distance from the tissue for the plurality of directions and/or punctiform spatial regions by means of the optical sensor and to provide the determined distances. To this end, the optical coherence tomography measuring device carries out an A-scan, by which a respective distance can be determined, in particular for each of the directions and/or spatial regions.

In particular, a surgical operating system is developed, comprising a surgical tool, an optical sensor arranged on or in the surgical tool and configured to measure a distance of the surgical tool, in particular of a distal end thereof, from a tissue for a plurality of directions and/or for a plurality of mutually separated punctiform spatial regions, an optical coherence tomography measuring device connected to the optical sensor and configured to determine the respective distance from the tissue for the plurality of directions and/or punctiform spatial regions by means of the sensor and to provide the determined distances.

Further, a surgical tool in particular is developed, comprising an optical sensor arranged on or in the surgical tool and configured to measure a distance of the surgical tool, in particular of a distal end thereof, from a tissue for a plurality of directions and/or for a plurality of mutually separated punctiform spatial regions.

Furthermore, a method for safeguarding a surgical operation in particular is made available, wherein a distance of a surgical tool, in particular of a distal end thereof, from a tissue is measured for a plurality of directions and/or for a plurality of mutually separated punctiform spatial regions by means of an optical sensor arranged on or in the surgical tool, wherein an optical coherence tomography measuring device connected to the optical sensor is used to determine a respective distance from the tissue for the plurality of directions and/or spatial regions using a sensor signal from the optical sensor as a starting point, and the determined distances are provided.

The term “a plurality of directions” should denote at least two directions in particular. For example, in this case “a plurality of directions” may comprise the following two directions: a direction in a direction of extent of the surgical tool from a proximal to a distal end, and a direction perpendicular thereto. In particular, in this case the “plurality of directions” may comprise a direction which extends away from an active or activatable tool region of the surgical tool from a perspective in front of said region. A “spatial region” should denote in particular a solid angle in which a measurement is implemented. In this case, mutually separated punctiform spatial regions should comprise in particular solid angles which do not have an intersection with one another, i.e. do not overlap one another.

The surgical operating system and the method can be used in the field of ophthalmology in particular. However, the surgical operating system can also be used in other fields of application, for example in ear, nose and throat surgery (e.g. during a cochlear implantation) or in neurosurgery.

Provision might be made for a measurement by means of the optical sensor to be preceded by carrying out one or more reference measurements, within the scope of which an A-scan is performed without an object (without tissue) in front of the optical sensor. An average value of this/these reference measurement(s) (A-scans) is then subtracted from the real measurement signal during later measurements, in order to thereby remove parasitic signal peaks from the measurement signal. It is also possible to divide the real A-scan by the average value of the reference measurement. Then, a simple peak detection algorithm in particular can be used within the scope of the measurement in order to identify the measurement signal. In particular, a simple threshold value detection can be performed to this end, in order to identify a relevant measurement signal. Depending on the field of application, the determinable distances in this case may lie in a range between 0 and a few millimeters (e.g. 2 mm, 4 mm or 8 mm). Further, provision can also be made for a pattern recognition to be performed, in order to identify a measurement signal. For example, the tissue, for example the retina, can be recognized with the aid of a pattern recognition (e.g. by way of template matching). Furthermore, machine learning methods and/or artificial intelligence methods might also be used to identify and/or evaluate the measurement signal provided by the optical sensor.

Provision might be made for a specified value (offset) to be subtracted from a determined distance, and for the corrected value to be provided as distance. As a result, it is possible to take account of more complex structures, in which it is not the outermost layer or surface closest to the sensor or the surgical tool that supplies the strongest measurement signal, but rather a layer located behind it. To take account of layer(s) situated in front when determining the distances, it is for example possible to take account of literature values for layer thicknesses.

Provision can be made for a position and/or an orientation of the surgical tool with respect to the tissue to be estimated using the determined distances as a starting point. Starting with two distances determined for different directions and/or spatial regions, an angle between the surgical tool and the surface of the tissue can be determined, for example with the aid of trigonometric considerations.

Further, provision can be made for a specularly and/or diffusely reflected intensity at the tissue to be evaluated in order to identify different tissue types. In this case, use can also be made of pattern recognition methods and/or machine learning and/or artificial intelligence methods.

Provision can be made for measurement signals from different directions and/or spatial regions to be retarded with the aid of at least one retardation path in an optical path. In this way, the measurement signals can be better distinguished from one another during the evaluation by the OCT measuring device. For example, a retardation path can be realized by means of a photonic integrated circuit.

Parts of the surgical operating system, in particular the OCT measuring device, can be embodied, either individually or together, as a combination of hardware and software, for example as program code that is executed on a microcontroller or microprocessor. However, it can also be provided that parts are designed, either individually or together, as an application-specific integrated circuit (ASIC) and/or a field-programmable gate array (FPGA). In particular, the OCT measuring device comprises at least one computing device and at least one memory. The computing device is configured in particular to execute program code stored in the memory for the purpose of carrying out the OCT measurement (evaluation, determination of the distances, etc.). The OCT measuring device can further comprise a display device and/or an operating device.

In an embodiment, provision is made for the optical sensor to comprise an optical fiber which is arranged on or in the surgical tool and at least one micro-optical element which, in particular at a distal end, is arranged and/or formed on the surgical tool on and/or in the fiber, with the at least one micro-optical element being configured to steer light from the optical fiber into the plurality of directions and/or into the plurality of mutually separated punctiform spatial regions and input couple light arriving from the plurality of directions and/or the spatial regions into the fiber and with the optical coherence tomography measuring device being connected to the optical fiber. The optical fiber allows particularly space-saving beam guidance from the optical coherence tomography measuring device to the surgical tool, in particular to the distal end of the surgical tool, and back. The at least one micro-optical element allows extremely space-saving beam shaping and beam conductance of the light guided through the optical fiber to the at least one micro-optical element. In particular, provision is made for the optical sensor to have a single optical fiber.

In an embodiment, provision is made for the surgical operating system to be configured to create and output feedback for a user using at least the determined distance with the smallest value as a starting point. As a result, the operating surgeon or assisting staff can be provided with feedback about the smallest, and hence in view of potential tissue damage most dangerous, distance determined. For example, feedback can be implemented visually, acoustically and/or haptically. For example, provision can be made for the provision of continuous feedback, within the scope of which for example a value of at least the distance with the smallest value is displayed as a numerical value and/or graphically. Furthermore, it is also possible to provide regions which are successively activated or deactivated depending on the determined distance. For example, a bar diagram with a plurality of such regions can be displayed, as known for park assistance systems in motor vehicles. The surgical operating system creates appropriate optical, acoustic and/or optical signals and supplies these to respective signal transmitters (e.g. display device, loudspeaker, force-feedback actuator, etc.).

In an embodiment, provision is made for the surgical operating system to be configured to compare the determined distances with at least one specified minimum distance and create and output at least one warning message and/or at least one warning signal and/or at least one control signal should the at least one specified minimum distance be undershot. Observance of a specified minimum distance can be monitored in a targeted manner as a result thereof. The warning message and/or the warning signal can be configured visually, acoustically and/or haptically. For example, the at least one control signal can be supplied to a robot controller which controls a robot assisting during the operation. In particular, the control signal can be a termination signal. Using the at least one created control signal as a starting point, the robot can for example suppress a further movement of the surgical tool or reverse an implemented movement, such that the specified minimum distance is observed again.

In an embodiment, provision is made for the surgical operating system to be configured to allow the specification of respective minimum distances for at least some of the plurality of directions and/or spatial regions, to compare the respective distances with the respective specified minimum distances, and to create and output at least one warning message and/or at least one warning signal and/or at least one control signal should the respective specified minimum distance be undershot. As a result, respective individual minimum distances can be specified and monitored for the directions and/or spatial regions.

In an embodiment, provision is made for the surgical tool to be an ophthalmological surgical tool. For example, a surgical tool can be a vitrectome, a light tube, an endoillumination tool, a needle, a gripper, a hook, a pair of forceps, a laser probe, a capsulotomy tool, an injector or a cannula.

In an embodiment, provision is made for the surgical operating system to be configured to measure and/or determine at least some of the respective distances simultaneously. As a result, the respective distances can all be provided at the same time. In particular, to this end, provision is made for light guided through the optical fiber for the individual directions and/or spatial regions to differ in terms of at least one property. As a result, the different directions and/or spatial regions can be separated from one another. In particular, a distance can be measured and determined simultaneously for each of the directions and/or spatial regions.

In an embodiment, provision is made for the optical sensor to be configured to guide light of different polarization directions into different directions and/or spatial regions of the plurality of directions and/or spatial regions and capture light arriving therefrom while creating different polarization directions for the different directions and/or spatial regions of the plurality of directions and/or spatial regions, with the optical coherence tomography measuring device being configured to assign the different directions and/or spatial regions on the basis of the polarization direction. In particular, provision is made for the at least one micro-optical element to be configured to guide light of different polarization directions from the fiber into different directions and/or spatial regions of the plurality of directions and/or spatial regions and input couple light arriving therefrom into the optical fiber while creating different polarization directions for the different directions and/or spatial regions of the plurality of directions and/or spatial regions. For example, suitable polarization filters can be used to this end.

In an embodiment, provision is made for the optical sensor to be configured to guide light from different wavelength ranges into different directions and/or spatial regions of the plurality of directions and/or spatial regions and to capture light arriving therefrom while maintaining the different wavelength ranges for the different directions and/or spatial regions of the plurality of directions and/or spatial regions, with the optical coherence tomography measuring device being configured to assign the different directions and/or spatial regions on the basis of the wavelength ranges. In particular, provision is made for the at least one micro-optical element to be configured to guide light of different wavelength ranges into different directions and/or spatial regions of the plurality of directions and/or spatial regions and input couple light arriving therefrom into the optical fiber while maintaining the different wavelength ranges for the different directions and/or spatial regions of the plurality of directions and/or spatial regions. For example, suitable prisms or diffraction gratings, etc., can be used to this end.

In an embodiment, provision is made for the optical sensor to be configured to provide respective light for the different directions and/or spatial regions of the plurality of directions and/or spatial regions with different OCT working distances, with the optical coherence tomography measuring device being configured to assign the different directions and/or spatial regions on the basis of the OCT working distances. In particular, provision is made for the optical fiber and the at least one micro-optical element to be configured to provide respective light for the different directions and/or spatial regions of the plurality of directions and/or spatial regions with different OCT working distances. In this case, the OCT working distance is, in particular, the distance between a portion relevant to the OCT measurement (i.e. the “OCT probe”) and the target region (i.e. the tissue in particular) for which the measured signal intensity is greatest. For example, this OCT working distance is defined by the focal length of the at least one micro-optical element on the surgical tool, in particular at the distal end of the surgical tool, and can be specified in each case for the different directions when designing the optical sensor. For different directions, signals with a sufficiently large signal amplitude are only expected within a predetermined range around the respective OCT working distance as a result of different OCT working distances. For example, one direction could have an OCT working distance of 1 mm with a corresponding measurement range from 0 to 2 mm, and another direction could have an OCT working distance of 4 mm with a corresponding measurement range of 2 mm to 6 mm. In this case, the signals can be separated because, for the respective distances, only the directions corresponding therewith can be the causal ones. Like the OCT working distance as well, the respective measurement range is defined during the design of the optical sensor.

In an embodiment, provision is made for the optical sensor to be configured to guide light of different intensities into different directions and/or spatial regions of the plurality of directions and/or spatial regions and capture light arriving therefrom, with the optical coherence tomography measuring device being configured to assign the different directions and/or spatial regions on the basis of the intensities. In particular, provision is made for the at least one micro-optical element to be configured to guide light of different intensities into different directions and/or spatial regions of the plurality of directions and/or spatial regions and input couple light arriving therefrom into the fiber. The optical sensor, in particular the at least one micro-optical element, can be configured to create the different intensities, for example by virtue of performing a suitable attenuation for the respective directions and/or spatial regions.

In an embodiment, provision is made for the optical sensor to be configured to guide light of different spectral widths into different directions and/or spatial regions of the plurality of directions and/or spatial regions and capture light arriving therefrom, with the optical coherence tomography measuring device being configured to assign the different directions and/or spatial regions on the basis of the different spectral widths. In particular, for example, signals of a laser source are created with different spectral widths. In particular, provision is made for the at least one micro-optical element to be configured to guide light of different spectral widths into different directions and/or spatial regions of the plurality of directions and/or spatial regions and input couple light arriving therefrom into the fiber.

In an embodiment, provision is made for the optical fiber to be a multi-mode fiber, with the at least one micro-optical element being configured to guide light of different modes from the optical fiber into different directions and/or spatial regions of the plurality of directions and/or spatial regions and input couple light arriving therefrom back into the respective original mode of the optical fiber, with the optical coherence tomography measuring device being configured to assign the different directions and/or spatial regions on the basis of the modes. As a result, a direction and/or a spatial region can be assigned to each mode. In this context, use can be made of spatial multiplexers which are based on phase plates or photonic lanterns, for example.

Provision can be made for the optical fiber to be a multi-core fiber, with the at least one micro-optical element being configured to guide light from different cores from the optical fiber into different directions and/or spatial regions of the plurality of directions and/or spatial regions and input couple light arriving therefrom back into the respective original core of the optical fiber, with the optical coherence tomography measuring device being configured to assign the different directions and/or spatial regions on the basis of the cores. As a result, a direction and/or a spatial region can be assigned to each core. An example of micro-optical elements arranged on a multi-core fiber is found in Philipp-Immanuel Dietrich et al., Printed freeform lens arrays on multi-core fibers for highly efficient coupling in astrophotonic systems, Optics Express vol. 25, no. 15, pp. 18288-18295 (2017) https://doi.org/10.1364/OE.25.018288. Further, Thorlabs, Inc., USA sells multi-core fibers which, for example, have a multi-core fiber at one end and a plurality of individual fibers at another end. The multi-core end can be used for the optical sensor, whereas the individual fibers are each connected to a detector of the OCT measuring device.

In an embodiment, provision is made for the operating system to be configured to temporally modulate light guided in different directions and/or spatial regions of the plurality of directions and/or spatial regions by means of the optical sensor, with the optical coherence tomography measuring device being configured to assign the different directions and/or spatial regions on the basis of the temporal modulation. In the simplest case, a modulation can be for example a modulation of the intensity (or pulse height or amplitude) of a pulsed signal. In that case, a pulse with a pulse height that changes over time in accordance with the modulation appears in the captured sensor signal. If different modulations are chosen, e.g. at different frequencies, then the individual directions and/or spatial regions can be distinguished from one another in the captured sensor signal and by the optical coherence tomography measuring device. In particular, the modulation is implemented in the optical coherence tomography measuring device. In addition to that or in an alternative, it is also possible as a matter of principle to perform the modulation in the optical sensor, in particular by means of one or more micro-optical elements.

In an embodiment, provision is made for at least some of the respective distances to be measured and/or determined sequentially. In particular, the distances for the plurality of directions and/or spatial regions are measured and/or determined in sequence, with this measurement being repeated cyclically in particular. In particular, each of the plurality of directions and/or each of the plurality of spatial regions is assigned a temporal measurement window within which light is steered into the respective direction or the respective spatial region and captured therefrom. As a result of the temporal relationship between the temporal measurement windows and the directions and/or spatial regions, it is possible to assign the direction or the spatial region to a captured sensor signal or measurement signal, and so a distance can be determined for each time window or each direction and/or each spatial region.

In an embodiment, provision is made for the surgical operating system to be configured to temporally sweep a wavelength of the light guided into the different directions and/or spatial regions, with the optical sensor being configured to successively guide the light, matched to the wavelength, into the different directions and/or spatial regions and to capture light arriving therefrom, with the optical coherence tomography measuring device being configured to assign the different directions and/or spatial regions in a manner matched to the wavelength. For example, a light source of the optical coherence tomography measuring device can be swept in a specified wavelength range with a specified frequency. In this case, a frequency is of the order of hundreds of kHz or even MHz, for example. By means of a micro-optical element, the light swept in this manner can be actively or passively guided into the different directions and/or spatial regions and light arriving from the different directions and/or spatial regions can be captured. In this case, too, provision can be made for a frequency used to modify a wavelength of the light to be greater than a frequency with which the optical sensor, in particular the at least one micro-optical element, changes the directions. As a result, a measurement signal created by means of light of different wavelengths can be obtained from each direction or each spatial region.

In an embodiment, provision is made for the optical sensor to comprise at least one dichroic mirror as a micro-optical element. As a result, a direction can be specified passively on the basis of a wavelength range of the light. A dichroic mirror reflects light from one wavelength range (λ1 to λ2) in one direction, whereas light from another wavelength range (λ2 to λ3) is passed (transmitted). In particular, this allows the development of a micro-optical element which reflects light from the one wavelength range at right angles, for example, and passes light from the other wavelength range in a straight line. By changing the wavelength of the light source, the direction or the spatial region in which the measurement is carried out can thus be changed. A simultaneous measurement of a plurality of directions or spatial regions is also possible if the received light spectrum is decomposed into the individual wavelength ranges (in the example λ1 to λ2 and λ2 to λ3) prior to a Fourier transform.

In an embodiment, provision is made for the optical sensor to comprise a switchable micro-optical element as micro-optical element, in which it is possible to set the direction and/or the spatial region into which the light is guided, with the surgical operating system being configured to control the switchable micro-optical element in such a way that the light is successively guided into the different directions and/or spatial regions and is captured therefrom again, with the optical coherence tomography measuring device being configured to assign the different directions and/or spatial regions in a manner matched to the control of the switchable micro-optical element. For example, the switchable micro-optical element can be an optical switch or a switchable mirror. For example, such a switchable mirror can be moved and/or tilted by means of capacitive, electromagnetic, piezoelectric or thermal actuation. Control enables the switchable micro-optical element to be brought into a specified position or angular position in order to guide light in a specified direction and/or into a specified spatial region and capture light from this direction and/or this spatial region. A movement of the micro-optical element, for example the switchable mirror, can be implemented about more than one axis in particular. Other examples of a switchable optical element include: moving the optical fiber for mechanically steering the beam and/or mechanically moving an optical lens for steering the beam; steering the beam in an acousto-optic and/or electro-optic fashion; steering the beam by means of a liquid crystal (refractively or diffractively); steering the beam by means of a switchable photonic array by using integrated photonics; steering a beam by means of an optical phased array (matched thermally or electro-optically); steering the beam by means of one or more liquid lenses; steering the beam by means of meta-materials.

In an embodiment, provision is made for the surgical tool to comprise a movable element, with the movable element being configured to be able to guide light into the different directions and/or spatial regions by means of a movement of the movable element and to be able to capture the said light from these directions and/or spatial regions, with the optical coherence tomography measuring device being configured to assign the different directions and/or spatial regions in a manner matched to the movement of the movable element. In particular, the movable element is an active manipulation and/or cutting element of the surgical tool. For example, in the case of a vitrectome, a movable element is a periodically moving blade of the vitrectome arranged on a window in the vicinity of a distal end of the vitrectome. For example, a micro-optical element can be arranged on this blade and steer the light from the optical fiber to the outside in the surroundings of the blade. As a result of the periodic movement of the blade, the surroundings are scanned by means of the deflected light in accordance with this movement. Then, taking account of the periodic movement or a control signal for causing the periodic movement, captured light can be evaluated such that the respective captured light can be assigned to different directions and/or spatial regions in the scanned surroundings.

In an embodiment, provision is made for the optical coherence tomography measuring device to be configured to take account of a position, an orientation and/or a movement of the surgical tool when determining the distances. This can further improve the determination of the distances. In particular, provision can be made for the determined distances to be recorded and tracked, in order to check the plausibility of subsequently determined values for the distances on the basis of the movements carried out by the surgical tool. In particular, consideration can be given to the position, the orientation and/or the movement of a robotic system which guides the surgical tool or assists a movement of the surgical tool. This can improve a robustness and a reliability of the optical sensor or the distance determination. For example, if a distinction between the directions and/or the spatial regions cannot be made on the basis of different pulse heights, the information from the robotic system regarding a position, an orientation and/or a movement of the surgical tool can be used to distinguish the pulses in the captured sensor signals and to assign these to the different directions or spatial regions. There are only two possible options in an example with two directions, with one of the options being plausible when the information from the robotic system is used as a starting point, whereas the other option is not plausible. Further, plausibility can be checked by virtue of a movement of the robotic system, for which movement information (extent of the movement along one or more axes) is provided by the robotic system, being compared with movement information derived from a change in the measured and determined distances, and a deviation between the items of movement information being assessed as an indication for a fault of the optical sensor. Provision can be made for an error message to be created and output by the OCT measuring device.

In an embodiment, provision is made for the optical coherence tomography measuring device to be configured to assign the directions and/or spatial regions taking into account a position, an orientation and/or a movement of the surgical tool. The movements of the surgical tool by means of a robotic system can be used in particular for distinguishing the different directions since a movement of the surgical tool along an axis always coincides with a change in distance in a direction associated therewith and/or in a spatial region associated therewith. If a distance changes when the surgical tool is moved along this axis, then the direction associated with the distance can be assigned to a direction extending along this axis.

In an embodiment, provision is made for the optical coherence tomography measuring device to be configured to take account of a model of an organ or body part when determining the distances. This allows the determination of a position of the surgical tool in relation to a geometric shape of the organ or body part, for example the eye, in the case of a sufficient number of measured and determined distances in the plurality of directions and/or spatial regions. If a continuous movement of the surgical tool is assumed, then a position of the surgical tool can be tracked in the organ or body part, in particular in the eye. If a continuous movement is assumed, it is moreover also possible to filter out signal fluctuations if these signal fluctuations are implausible with regard to a continuous movement. Further, it is also possible to distinguish between different distance signals in this way.

In an embodiment, provision is made for the optical coherence tomography measuring device to be configured to take account of at least one calibration curve when determining the distances. As a result, individual properties of the optical sensor can be taken into account during the evaluation. Prior to the use of the surgical operating system, the at least one calibration curve can be determined by means of suitable calibration methods and/or test objects.

In an embodiment, provision is made for the optical sensor to be configured so that at least one spectral property of light guided into different directions and/or spatial regions differs, with the optical coherence tomography measuring device being configured to assign the different directions and/or spatial regions while taking account of the respective spectral properties.

In an embodiment, provision is made for the spectral properties to comprise at least one of the following: a signal width, a distance between a signal and a parasitic signal, a property of a parasitic signal.

Provision can be made for the embodiments described in this disclosure to be realized individually or in combination with one another.

Features relating to the configuration of the surgical tool arise from the description of configurations of the surgical operating system. The advantages of the surgical tool here are in each case the same as for the configurations of the surgical operating system.

Further features relating to the configuration of the method are evident from the description of configurations of the surgical operating system. Here, the advantages of the method are in each case the same as in the configurations of the surgical operating system.

The invention is explained in greater detail below on the basis of preferred exemplary embodiments with reference to the figures. In the figures:

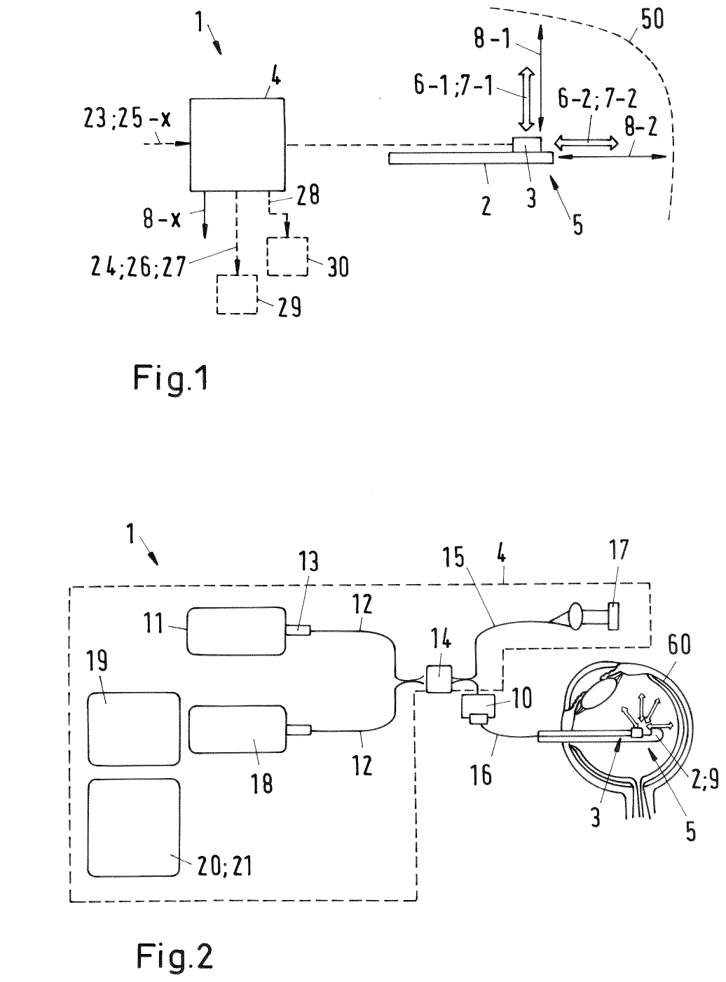

FIG. 1 shows a schematic illustration for elucidating an embodiment of the surgical operating system;

FIG. 2 shows a schematic illustration for elucidating an embodiment of the surgical operating system;

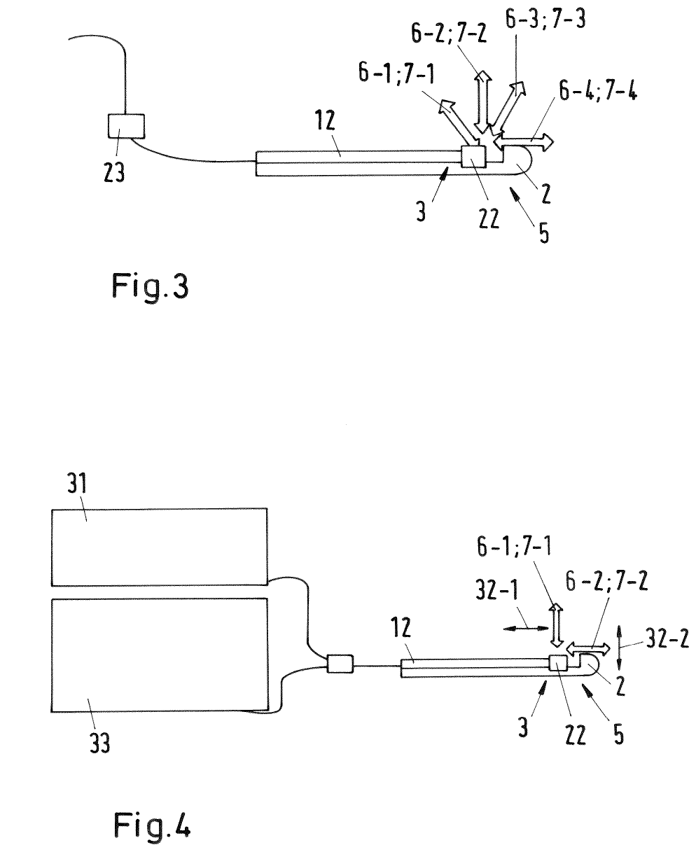

FIG. 3 shows a schematic illustration for elucidating an embodiment of the surgical operating system;

FIG. 4 shows a schematic illustration for elucidating an embodiment of the surgical operating system;

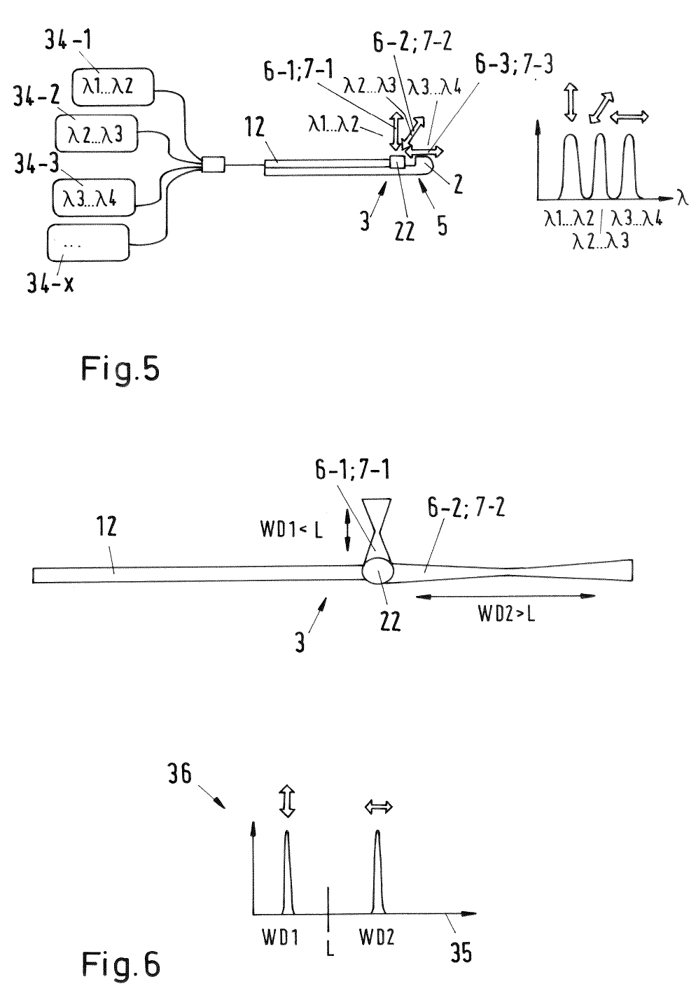

FIG. 5 shows a schematic illustration for elucidating an embodiment of the surgical operating system;

FIG. 6 shows a schematic illustration for elucidating an embodiment of the surgical operating system;

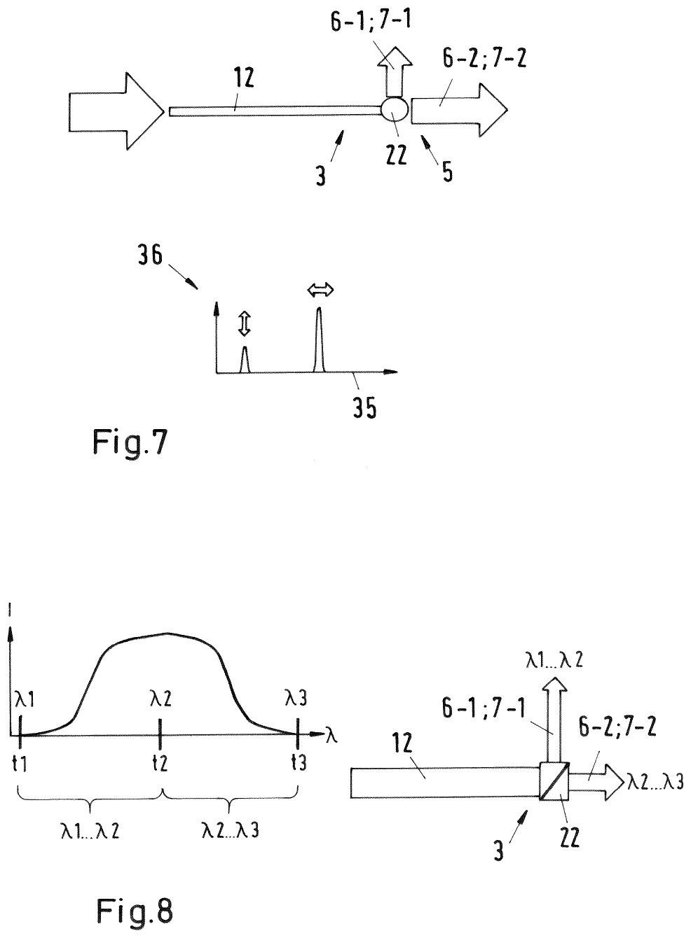

FIG. 7 shows a schematic illustration for elucidating an embodiment of the surgical operating system;

FIG. 8 shows a schematic illustration for elucidating an embodiment of the surgical operating system;

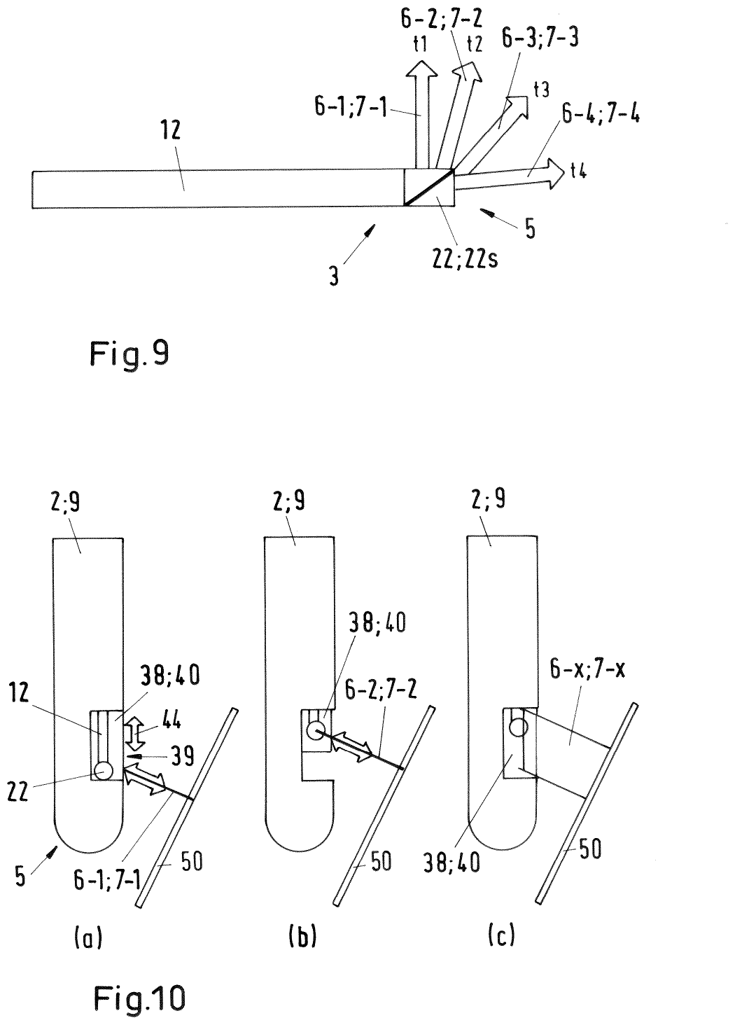

FIG. 9 shows a schematic illustration for elucidating an embodiment of the surgical operating system;

FIGS. 10a-c show schematic illustrations for elucidating an embodiment of the surgical operating system;

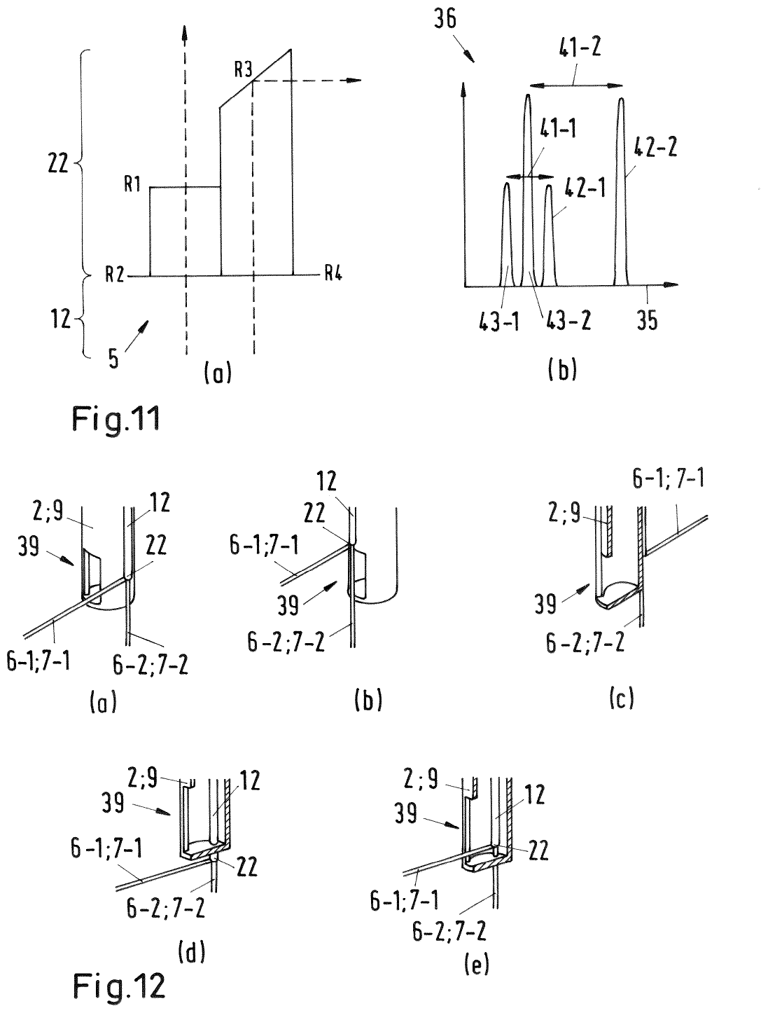

FIGS. 11a, b show a schematic illustration for elucidating an embodiment of the surgical operating system;

FIGS. 12a-e show schematic illustrations for elucidating embodiments of a surgical operating system.

FIG. 1 shows a schematic illustration for elucidating an embodiment of the surgical operating system 1. The surgical operating system 1 comprises a surgical tool 2, an optical sensor 3 and an optical coherence tomography (OCT) measuring device 4.

The optical sensor 3 is arranged on or in the surgical tool 2. The optical sensor 3 is configured to measure a distance 8-x of the surgical tool 2, in particular of a distal end 5 thereof, from a tissue 50 for a plurality of directions 6-x and/or for a plurality of mutually separated punctiform spatial regions 7-x. This is shown for two mutually perpendicular directions 6-x and/or spatial regions 7-x by way of example. However, more than two directions 6-x and/or spatial regions 7-x can also be provided. The directions 6-x and/or spatial regions 7-x can also be aligned differently with respect to one another.

The OCT measuring device 4 is connected to the optical sensor 3. The OCT measuring device 4 is configured to determine the respective distance 8-x from the tissue 50 for the plurality of directions 6-x and/or punctiform spatial regions 7-x by means of the optical sensor 3 and to provide the determined distances 8-x. To this end, the OCT measuring device 4 performs a respective A-scan, in particular, for the plurality of directions 6-x and/or spatial regions 7-x, by means of which it is possible in a manner known per se to determine a distance 8-x from a surface (a tissue layer of the tissue 50, in particular, in the present case) by virtue of a specular and/or diffuse reflection of light radiated onto the surface being superimposed on light from a reference path and thus being made to interfere therewith. The respective distance 8-x determined thus is provided, more particularly output, as an analog or digital distance signal, for example.

The surgical tool 2 of the surgical operating system 1 can be guided by the hand of a surgeon. As an alternative to that or in addition, the surgical tool 2 can also be guided by a robotic system 30, in order to assist a surgeon during the operation. In particular, the surgical operating system 1 can also be part of, or comprise, a robotic system 30.

In particular, provision can be made for the surgical tool 2 to be an ophthalmological surgical tool 2. However, the surgical operating system 1 can in principle also be used in other fields of application provided that distances can be determined optically.

FIG. 2 shows a schematic illustration for elucidating an embodiment of the surgical operating system 1, in which the basic setup of the OCT measuring device 4 is depicted in more detail. As surgical tool 2, the example shows a vitrectome 9 which is controlled by way of a vitrectome controller 10. The vitrectome 9 is introduced into the vitreous humor of the eye 60 by way of a trocar and used there to remove portions of the vitreous humor (vitrectomy). Distance monitoring is desired for safety reasons should work be performed in the vicinity of the retina.

The OCT measuring device 4 comprises a coherent light source 11, for example a laser. The light from the light source 11 is input coupled into an optical fiber 12 by means of a connector 13 and guided in two optical arms 15, 16 (this is referred to as a dual path), a reference arm 15 and a measuring arm 16, by way of a beam splitter 14. In principle, the reference arm 15 can also be integrated in the measuring arm 16 (this is referred to as a common path). A mirror 17 which guides the light back to the beam splitter 14 is arranged at the end of the reference arm 15. An optical path length that the light travels through the reference arm 15 can be set by modifying a position of the mirror 17. The light that was guided in the measuring arm 16 is guided to the distal end 5 of the surgical tool 2 and, from there, in the plurality of directions 6-x and/or spatial regions 7-x. Light reflected by a surface, in particular a tissue 50 such as the retina in the present case, is input coupled back into the measuring arm 16 coming from the plurality of directions 6-x and/or spatial regions 7-x and guided back to the beam splitter 14. The light guided back from the reference arm 15 and the measuring arm 16 interferes in the beam splitter 14. The result of the interference in the beam splitter 14 is guided to a detector 18 by means of an optical fiber 12 and a connector 13 and is captured by the said detector. A detector signal is evaluated in a manner known per se (OCT) using a signal processing device 19, and the measurement results of the optical sensor 3 from the plurality of directions 6-x and/or spatial regions 7-x are separated and/or distinguished from one another, and a distance from the tissue 50 (from the retina in the present example) is determined for each of the directions 6-x and/or spatial regions 7-x. The respective determined distances are provided; for example, these are displayed on a display device 20 or feedback that depends on a respective distance is provided for the surgeon and/or another user, for example by suitable signal transmitters 21.

The OCT measuring device 4 can apply different methods as a matter of principle: time-domain OCT, spectral-domain OCT or swept-source OCT. A configuration can have a common-path (CP) embodiment or, as shown in FIG. 2 by way of example, a dual-arm (DA) embodiment. In any case, only one-dimensional depth scans (A-scans) are carried out in order to thus create a respective one-dimensional depth map for each of the directions 6-x and/or spatial regions 7-x, from which the respective distance can subsequently be determined (the respective distance corresponds to the respective depth determined from the depth map in this case).

FIG. 3 shows a schematic illustration for elucidating an embodiment of the surgical operating system. In this embodiment, provision is made for the optical sensor 3 to comprise an optical fiber 12 which is arranged on or in the surgical tool 2 and at least one micro-optical element 22 which is arranged and/or formed on the surgical tool 2, in particular at a distal end 5, on and/or in the fiber 12, with the at least one micro-optical element 22 being configured to steer light from the optical fiber 12 into the plurality of directions 6-x and/or into the plurality of mutually separated punctiform spatial regions 7-x and input couple light arriving from the plurality of directions 6-x and/or the spatial regions 7-x into the fiber 12 and with the optical coherence tomography measuring device 4 being connected to the optical fiber 12. For example, the connection to the OCT measuring device 4 is implemented by way of a fiber coupler 23. In particular, the optical fiber 12 is a single-mode fiber. In principle, however, other optical fibers 12 (multi-mode, multi-core, polarization-maintaining, etc.) can also be used. In particular, the optical fiber 12 extends to an active region of the surgical tool 2, where a manipulation or cutting element, for example, is arranged. In particular, the optical fiber 12 extends to a distal end 5 of the surgical tool 2.

To guide light from the optical fiber 12 into the plurality of directions 6-x and/or spatial regions 7-x and to capture the said light therefrom, the at least one micro-optical element 22 can for example comprise one or more of the following elements: a gradient index (GRIN) lens, a ball lens, three dimensionally printed lenses, a polarizing beam splitter, a dichroic mirror, a diffraction beam splitter, a diffraction grating, an optical grating. Further, the at least one micro-optical element 22 can also be designed as a part of the optical fiber 12, for example in the form of a cleaved, angled or angle-polished fiber end, in order thereby to (partially) guide the light from the fiber into the different directions 6-x and/or spatial regions 7-x. Provision can also be made for surfaces of the optical fiber 12 to be coated. For example, provision can be made for an end facet of the optical fiber 12 and/or for a side or a region of the micro-optical element 22 to be coated, for example with a thin gold layer. This can create a particularly good reflection at this layer as a reference signal in a common-path (CP) configuration of the OCT measuring device 4.

Provision can be made for the surgical operating system 1 to be configured to create and output feedback 24 (FIG. 1) for a user using at least the determined distance 8-x with the smallest value as a starting point. In the simplest case, the surgical operating system 1 outputs the respective current value of the shortest distance 8-x. For example, the value can be displayed on a display device 20 (FIG. 2). The values for all determined distances can also be output.

Provision can be made for the surgical operating system 1 to be configured to compare the determined distances 8-x with at least one specified minimum distance 25 (FIG. 1) and create and output at least one warning message 26 and/or at least one warning signal 27 and/or at least one control signal 28 should the at least one specified minimum distance 25 be undershot. For example, the minimum distance 25 can be specified by a user by way of an operating device (not shown) of the surgical operating system 1. The at least one warning message 26 and/or the at least one warning signal 27 can have a visual, acoustic and/or haptic configuration and are supplied to a respective suitable signal transmitter 29 of the surgical operating system 1 in particular. For example, the at least one control signal 28 can be supplied to a robotic system 30 which guides the surgical tool 2 and which can also be part of the surgical operating system 1. Using the at least one control signal 28 as a starting point, the robotic system 30 can for example stop and/or reverse a currently performed movement of the surgical tool 2.

Provision can be made for the surgical operating system 1 to be configured to allow the specification of respective minimum distances 25-x (FIG. 1) for at least some of the plurality of directions 6-x and/or spatial regions 7-x, to compare the respective distances 8-x with the respective specified minimum distances 25-x, and to create and output at least one warning message 26 and/or at least one warning signal 27 and/or at least one control signal 28 should the respective specified minimum distance 25-x be undershot. In this context, the procedure is the same as already described above. This embodiment allows monitoring of an individually specified minimum distance 25-x for each of the directions 6-x and/or spatial regions 7-x and in particular provision of a directionally dependent or spatial region-dependent warning message 26 and/or warning signal 27.

Provision can be made for at least some of the respective distances 8-x to be measured and/or determined at the same time. To this end, the surgical operating system 1 performs signal multiplexing, in particular, in order to be able to separate the measurement signals from different directions 6-x and/or spatial regions 7-x from one another. In particular, different properties of the light can be used in this respect in order to measure the different directions 6-x and/or spatial regions 7-x, for example different polarizations, different wavelengths (wavelength ranges), different intensities, different OCT working distances (spatial multiplexing), different spectral widths, different fiber modes and using a signal modulation.

FIG. 4 shows a schematic illustration for elucidating an embodiment of the surgical operating system 1, in which different polarizations are used to distinguish the directions 6-x and/or spatial regions 7-x from one another in the case of a simultaneous measurement. In this embodiment, provision is made for the optical sensor 3 to be configured to guide light of different polarization directions into different directions 6-x and/or spatial regions 7-x of the plurality of directions and/or spatial regions and capture light arriving therefrom while creating different polarization directions for the different directions 6-x and/or spatial regions 7-x of the plurality of directions and/or spatial regions, with the optical coherence tomography measuring device 4 (FIG. 1) being configured to assign the different directions 6-x and/or spatial regions 7-x on the basis of the polarization direction. In particular, the optical sensor 3 comprises a polarization-maintaining optical fiber 12 and at least one micro-optical element 22. Within the OCT measuring device, a suitable optical element 31 is used to create a number x of mutually orthogonal polarization modes and input couple these into the polarization-maintaining optical fiber 12 of the optical sensor 3. For example, the micro-optical element 22 of the optical sensor 3 comprises a polarizing beam splitter (PBS) at the distal end of the optical fiber 12 and, as a result thereof, guides the polarized light in the different directions 6-x and/or spatial regions 7-x (two directions/spatial regions perpendicular to one another are shown by way of example). For example, the polarizing beam splitter can operate with the aid of metasurfaces (examples of such a beam splitters are found in: Sheng Li Jia et al., Independent controls of orthogonally polarized transmitted waves using a Huygens metasurface, vol. 9, no. 5, September 2015, pp. 545-553, https://doi.org/10.1002/lpor.201500094; and Zhe Shen and Dingxin Huang, A Review on Metasurface Beam Splitters, Nanomanufacturing 2022, 2 (4), pp. 194-228, https://doi.org/10.3390/nanomanufacturing2040014). The E-field vectors 32-x of the (two exemplary) directions 6-x and/or spatial regions 7-x are perpendicular to one another. The light arriving from the two directions 6-x and/or spatial regions 7-x also has the same directions for the E-field vectors 32-x. The incident light is input coupled back into the optical fiber 12 with the same polarization as that of the original light and is guided to a suitable further optical element 33 (polarization filter), decomposed into the signal constituents there on the basis of the polarization, and measured by means of a respective detector (not shown) for each polarization state. The evaluation for determining the respective distances for the directions 6-x and/or spatial regions 7-x is then implemented as described above.

FIG. 5 shows a schematic illustration for elucidating an embodiment of the surgical operating system 1, in which different wavelengths (wavelength ranges) are used to distinguish the directions 6-x and/or spatial regions 7-x from one another in the case of a simultaneous measurement. In this embodiment, provision is made for the optical sensor 3 to be configured to guide light from different wavelength ranges into different directions 6-x and/or spatial regions 7-x of the plurality of directions and/or spatial regions and to capture light arriving therefrom while maintaining the different wavelength ranges for the different directions 6-x and/or spatial regions 7-x of the plurality of directions and/or spatial regions, with the optical coherence tomography measuring device 4 (FIG. 1) being configured to assign the different directions 6-x and/or spatial regions 7-x on the basis of the wavelength ranges. In particular, the optical sensor 3 comprises an optical fiber 12 and at least one micro-optical element 22 to this end. For example, the OCT measuring device 4 comprises a plurality of light sources 34-x, which each create light from a different wavelength range (by way of example, three wavelength ranges are specified explicitly as λ1 to λ2, λ2 to λ3 and λ3 to λ4). In principle, a single broadband light source whose light is split into the wavelength ranges might also be provided. The light is input coupled into the optical fiber 12 for the plurality of wavelength ranges. Then, the at least one optical element 22 guides each of the wavelength ranges in a different direction 6-x and/or to a different spatial region 7-x and also captures incident light in the respective wavelength range only from the assigned direction 6-x and/or from the assigned spatial region 7-x and output couples the said light into the optical fiber 12 while maintaining the respective wavelength range. In the OCT measuring device 4 (FIG. 1), the light is split into the wavelength ranges and guided to a respective detector (not shown). As a result, a dedicated measurement signal from which the respective distance can be determined is available for each wavelength range, and hence for each direction 6-x and/or each spatial region 7-x. For example, a guidance of different wavelength ranges into different directions 6-x and/or spatial regions 7-x at the distal end 5 of the surgical tool 2 can be implemented using at least one of the following optical elements, in particular as part of the at least one micro-optical element 22: a miniaturized diffractive optical element (DOE) which creates wavelength-dependent diffraction angles; an optical element having total-internal reflection (TIR), for example a prism with TIR; a dispersive prism; a diffraction grating or optical grating; a diffraction lens; a dichroic mirror or wavelength separator which exhibits strong reflection at one wavelength and transmission at another wavelength such that a split into two beams is possible; a transmission or reflection volume hologram (cf. Flavio Guerra et al., Holographic Wide-Angle System for Deformation Measurement of Extended Structures, Optics 2022, 3(1), 79-87; https://doi.org/10.3390/opt3010010; use in the reverse beam direction is possible here in the same way); various bandpass filters for the various wavelengths (wavelength ranges).

FIG. 6 shows a schematic illustration for elucidating an embodiment of the surgical operating system 1, in which different OCT working distances are used to distinguish the directions 6-x and/or spatial regions 7-x from one another in the case of a simultaneous measurement. In this embodiment, provision is made for the optical sensor 3 to be configured to provide respective light for the different directions 6-x and/or spatial regions 7-x of the plurality of directions and/or spatial regions with different OCT working distances WD1, WD2, with the optical coherence tomography measuring device 4 (FIG. 1) being configured to assign the different directions 6-x and/or spatial regions 7-x on the basis of the OCT working distances WD1, WD2. In particular, this achieves different optical retardations. In particular, the optical sensor 3 comprises an optical fiber 12 and at least one micro-optical element 22 to this end. The at least one micro-optical element 22 creates a plurality of light beams (two in the example shown) and guides these into different directions 6-x and/or spatial regions 7-x. On account of the various OCT working distances WD1, WD2, the measurement signals for the working distances WD1, WD2 appear at different depths 35 in the one-dimensional depth map 36 (cf. inset in FIG. 6). This embodiment can be further improved if the distances determined from the measurement signals are tracked over time and the plausibility thereof is checked on the basis of previous values.

FIG. 7 shows a schematic illustration for elucidating an embodiment of the surgical operating system 1, in which different intensities I are used to distinguish the directions 6-x and/or spatial regions 7-x from one another in the case of a simultaneous measurement. In this embodiment, provision is made for the optical sensor 3 to be configured to guide light of different intensities I into different directions 6-x and/or spatial regions 7-x of the plurality of directions and/or spatial regions and capture light arriving therefrom, with the optical coherence tomography measuring device 4 (FIG. 1) being configured to assign the different directions 6-x and/or spatial regions 7-x on the basis of the intensities I. In this context, the assumption is made that, in particular, a specular and/or diffuse reflection at a tissue has no influence, or only a negligible influence, on an intensity difference. In particular, the optical sensor 3 comprises an optical fiber 12 and at least one micro-optical element 22 to this end. The at least one micro-optical element 22 creates a plurality of light beams (two in the example shown) with different intensities I (in FIG. 7, the intensity I is elucidated by an arrow size in each case and shown in the inset) and guides these into different directions 6-x and/or spatial regions 7-x. The measurement signals can be identified in the depth map 36 (cf. inset in FIG. 7) on account of the different intensities I. To this end, the at least one micro-optical element 22 can for example comprise a geometric beam splitter or a diffractive or refractive optical microstructure at the distal end 5 of the optical fiber 12 or a transmission volume hologram. This embodiment can be further improved if the distances determined from the measurement signals are tracked over time and the plausibility thereof is checked on the basis of previous values.

Provision can be made for the optical sensor 3 to be configured to guide light of different spectral widths into different directions 6-x and/or spatial regions 7-x of the plurality of directions and/or spatial regions and capture light arriving therefrom, with the optical coherence tomography measuring device 4 being configured to assign the different directions 6-x and/or spatial regions 7-x on the basis of the different spectral widths. In particular, the optical sensor 3 comprises an optical fiber 12 and at least one micro-optical element 22 to this end.

Provision can be made for the optical sensor 3 to comprise an optical fiber 12 and at least one micro-optical element 22, with the optical fiber 12 being a multi-mode fiber, with the at least one micro-optical element 22 being configured to guide light of different modes from the optical fiber 12 into different directions 6-x and/or spatial regions 7-x of the plurality of directions and/or spatial regions and input couple light arriving therefrom back into the respective original mode of the optical fiber 12, with the optical coherence tomography measuring device 4 being configured to assign the different directions 6-x and/or spatial regions 7-x on the basis of the modes.

Provision can be made for the optical sensor 3 to comprise an optical fiber 12 and at least one micro-optical element 22, with the optical fiber 12 being a multi-core fiber, with the at least one micro-optical element 22 being configured to guide light from different cores from the optical fiber 12 into different directions 6-x and/or spatial regions 7-x of the plurality of directions and/or spatial regions and input couple light arriving therefrom back into the respective original cores of the optical fiber 12, with the optical coherence tomography measuring device 4 (FIG. 1) being configured to assign the different directions 6-x and/or spatial regions 7-x on the basis of the cores.

Provision can be made for the surgical operating system 1 to be configured to temporally modulate light guided in different directions 6-x and/or spatial regions 7-x of the plurality of directions and/or spatial regions by means of the optical sensor 3, with the optical coherence tomography measuring device 4 (FIG. 1) being configured to assign the different directions 6-x and/or spatial regions 7-x on the basis of the temporal modulation.

In an alternative to that or in addition, provision can also be made for at least some of the respective distances 8-x to be measured and/or determined sequentially. To this end, the surgical operating system 1 performs temporal signal multiplexing, in particular, in order to be able to separate the measurement signals from different directions 6-x and/or spatial regions 7-x from one another. In this case, the measurements for the individual directions 6-x and/or spatial regions 7-x are implemented successively in time in particular, i.e., in particular, each individual direction 6-x and/or each spatial region 7-x is assigned a time window within which the measurement takes place.

FIG. 8 shows a schematic illustration for elucidating an embodiment of the surgical operating system 1 with a sequential measurement. In this embodiment, provision is made for the surgical operating system 1 to be configured to temporally sweep a wavelength of the light guided into the different directions 6-x and/or spatial regions 7-x, with the optical sensor 3 being configured to successively guide the light, matched to the wavelength, into the different directions 6-x and/or spatial regions 7-x and to capture light arriving therefrom, with the optical coherence tomography measuring device 4 (FIG. 1) being configured to assign the different directions 6-x and/or spatial regions 7-x in a manner matched to the wavelength. In particular, the optical sensor 3 comprises an optical fiber 12 and at least one micro-optical element 22 to this end. In particular, the OCT measuring device 4 (FIG. 1) comprises a sweepable light source, for example a sweepable laser, which is periodically swept from a wavelength λ1 (time t1) to a wavelength λ3 (time t2) (cf. inset in FIG. 8, in which an intensity I is plotted over wavelength λ and over the course of time). The at least one micro-optical element 22 guides the wavelength range λ1 to λ2 into one direction 6-1 and/or one spatial region 7-1 and the wavelength range λ2 to λ3 into another direction 6-2 and/or spatial region 7-2. Thus, wavelengths λ1 to λ2 are guided into the direction 6-1 and/or the spatial region 7-1 between the times t1 and t2, whereas the wavelengths λ2 to λ3 are guided into the direction 6-2 and/or the spatial region 6-2 between the times t2 and t3. In both time windows t1 to t2 and t2 to t3, light specularly and/or diffusely reflected at the tissue is coupled back into the optical fiber 12. The light input coupled into the fiber between the times t1 and t2 therefore originates from the direction 6-1 and/or the spatial region 7-1, and the light input coupled into the optical fiber 12 between the times t2 and t3 thus originates from the direction 6-2 and/or the spatial region 7-2. Since the (periodic) times t1, t2 and t3 are known, the OCT measuring device 4 (FIG. 1) is able to appropriately assign the directions 6-x and/or spatial regions 7-x on the basis of these time windows and determine the respective distances 8-x.

As an alternative to a sweepable light source, it is also possible to use a broadband light source and a tunable filter. It is also possible to use a broadband light source with two fixed different bandpass filters. If the spectrum is separated before a Fourier transform is performed, then it is even possible to measure both directions simultaneously.

To this end, provision can be made for the optical sensor 3 to comprise at least one dichroic mirror as a micro-optical element 22. Alternatively, use can also be made of a diffraction grating or a diffraction lens.

FIG. 9 shows a schematic illustration for elucidating a further embodiment of the surgical operating system 1 with a sequential measurement. In this embodiment, provision is made for the optical sensor 3 to comprise a switchable micro-optical element 22s as micro-optical element 22, in which it is possible to set the direction 6-x and/or the spatial region 7-x into which the light is guided, with the surgical operating system 1 being configured to control the switchable micro-optical element 22s in such a way that the light is successively guided into the different directions 6-x and/or spatial regions 7-x and is captured therefrom again, with the optical coherence tomography measuring device 4 (FIG. 1) being configured to assign the different directions 6-x and/or spatial regions 7-x in a manner matched to the control of the switchable micro-optical element 22s. By way of example, FIG. 9 shows four positions or angulations of the switchable micro-optical element 22s, which was set (periodically) at the times t1, t2, t3 and t4. The positions or angulations are maintained for a predetermined duration, with light from the optical fiber 12 being guided into the associated direction 6-x and/or into the associated spatial region 7-x and incident light from this direction 6-x and/or this spatial region 7-x being captured in a time window accompanying the said duration. Since the times t1, t2, t3 and t4 are periodically always the same, the light input coupled into the optical fiber 12 can be assigned to the individual directions 6-x or spatial regions 7-x.

FIG. 10 shows a schematic illustration for elucidating a further embodiment of the surgical operating system 1 with a sequential measurement. In this embodiment, provision is made for the surgical tool 2 to comprise a movable element 38, with the movable element 38 being configured to guide light into the different directions 6-x and/or spatial regions 7-x by means of a movement of the movable element 38 and to capture the said light from these directions 6-x and/or spatial regions 7-x, with the optical coherence tomography measuring device 4 (FIG. 1) being configured to assign the different directions 6-x and/or spatial regions 7-x in a manner matched to the movement of the movable element 38. In the example shown, the surgical tool 2 is a vitrectome 9. At the distal end 5, the vitrectome 9 comprises a window 39 in which a movable blade 40 (in particular in the form of a hollow cylinder) is arranged, the latter implementing a periodic movement between a lower position (FIG. 10a) (in relation to the illustration) and an upper position (FIG. 10b) (indicated by the double-headed arrow 44 in FIG. 10a) and thereby being able to be used to remove parts of the vitreous humor in the eye (vitrectomy). The optical sensor 3 comprises an optical fiber 12, which is guided in the interior of the surgical tool 2. For example, a micro-optical element 22 (e.g. a ball lens or a mirror) which deflects light from the fiber 12 out of the window 39 (FIG. 10a) is arranged at the distal end of the optical fiber 12. Together with the micro-optical element 22, the optical fiber 12 is arranged on the movable element 38 such that the distal end 5 of the optical fiber 12 and the micro-optical element 22 move between the lower position (FIG. 10a) and the upper position (FIG. 10b) together with the movable element 38. As a result of this movement, the deflected light is guided (scanned) over a plurality of directions 6-x and/or over a plurality of spatial regions 7-x, as depicted schematically in FIG. 10c. The movement is synchronous with a periodic control signal which controls the movable element 38. Using this control signal as a starting point, the OCT measuring device 4 (FIG. 1) can identify and assign the respective directions 6-x and/or spatial regions 7-x, and can as a result determine a respective distance for each direction 6-x and/or each spatial region 7-x.

Provision can be made for the optical coherence tomography measuring device 4 (FIG. 1) to be configured to take account of a position, an orientation and/or a movement of the surgical tool 2, preferably by means of at least one acceleration sensor, when determining the distances 8-x. In particular, this can be used to perform a check of the plausibility of the distances 8-x determined, by virtue of the determined distances 8-x being compared with distances 8-x which were determined previously and tracked over time.

Provision can be made for the optical coherence tomography measuring device 4 (FIG. 1) to be configured to take account of a model of an organ or body part when determining the distances 8-x. In particular, this can be used to perform a check of the plausibility of the distances 8-x determined, by virtue of the determined distances 8-x being compared with distances derived from the model. To this end, in particular a position and orientation of the surgical tool 2 within the organ (e.g. in the eye) or body part needs to be known. Further, data from a robotic system 30 can also be taken into account when checking the plausibility of determined distances 8-x and changes in the determined distances 8-x.

Provision can be made for the optical coherence tomography measuring device 4 (FIG. 1) to be configured to take account of at least one calibration curve when determining the distances 8-x. This can improve accuracy when determining the distances 8-x.

Provision can be made for the optical sensor 3 to be configured so that at least one spectral property of light guided into different directions 6-x and/or spatial regions 7-x differs, with the optical coherence tomography measuring device 4 (FIG. 1) being configured to assign the different directions 6-x and/or spatial regions 7-x while taking account of the respective spectral properties.

In particular, provision can be made for the spectral properties to comprise at least one of the following, cf. FIG. 11: a signal width, a distance 41-x between a signal 42-x and a parasitic signal 43-x, a property of a parasitic signal. For example, provision can be made for a signal width of light guided into different directions 6-x and/or spatial regions 7-x and captured therefrom to be selected differently. Then, the captured measurement signals can be distinguished from one another using the different signal widths as a starting point and can be assigned to the respective directions 6-x and/or spatial regions 7-x.

The use of different distances 41-x between a signal 42-x and a parasitic signal 43-x is elucidated schematically on the basis of FIG. 11. The distances 41-x in the depth map 36 (FIG. 11b) are specified here for example with the aid of a micro-optical element 22 (FIG. 11a), which is configured to this end and arranged at the distal end 5 of an optical fiber 12. The micro-optical element 22 has two different end facets for output coupling one part of the light in an axial direction and another part of the light in a lateral direction. For both parts, the micro-optical element 22 has different wavelengths, and so the optical path lengths of the parts of the light are different. The distance 41-1 is specified by an optical distance between the reflection surfaces R1 and R2, and the distance 41-2 is specified by the optical distance between the reflection surfaces R3 and R4, with the optical path length R1-R2 being different (smaller in this example) to the optical path length R3-R4. As a result of the distances 41-x, the associated signals 43-x can be assigned to the directions 6-x and/or spatial regions 7-x (in relation to the optical fiber 12 in the example, in particular an axial direction and a lateral direction). Provision can also be made for more than only two directions 6-x and/or spatial regions 7-x to be provided.

FIG. 12 shows schematic illustrations for elucidating embodiments of a surgical operating system, in which the surgical tool 2 is a vitrectome 9. The optical sensor 3 always comprises an optical fiber 12 and at least one micro-optical element 22.

In the embodiment shown in FIG. 12a, provision is made for the optical fiber 12 to be guided laterally on the outside of the vitrectome 9 and for the at least one micro-optical element 22 to steer the light from the optical fiber 12 into a direction 6-1 and/or spatial region 7-1 perpendicular to the window 39 and into the axial direction (into the distal direction of the surgical tool 2) and to capture light from these directions 6-x and/or spatial regions 7-x.

In the embodiment shown in FIG. 12b, provision is made for the optical fiber 12 to be guided as far as the window 39 on the outside of the vitrectome 9 on the side of the window 39 and for the at least one micro-optical element 22 to steer the light from the optical fiber 12 into a direction 6-1 and/or spatial region 7-1 perpendicular to the window 39 and into the axial direction (into the distal direction of the surgical tool 2) and to capture light from these directions 6-x and/or spatial regions 7-x.

In the embodiment shown in FIG. 12c, provision is made for the optical fiber 12 to be guided laterally on the outside of the vitrectome 9 on a side distant from the window 39 and for the at least one micro-optical element 22 to steer the light from the optical fiber 12 into a direction 6-1 and/or spatial region 7-1 perpendicular to the window 39 and toward the back and into the axial direction 6-2 (into the distal direction of the surgical tool 2) up to an axial spatial region 7-2 and to capture light from these directions 6-x and/or spatial regions 7-x.

In the embodiment shown in FIG. 12d, provision is made for the optical fiber 12 to be guided in the interior of the vitrectome 9 to a distal end of the surgical tool 2 and through a cutout at the distal end and for the at least one micro-optical element 22 to steer the light from the optical fiber 12 into a direction 6-1 and/or spatial region 7-1 perpendicular to the window 39 and into the axial direction 6-2 and/or into an axial spatial region 7-2 (into the distal direction of the surgical tool 2) and to capture light from these directions 6-x and/or spatial regions 7-x.

In the embodiment shown in FIG. 12e, provision is made for the optical fiber 12 to be guided in the interior of the vitrectome 9 almost to a distal end of the surgical tool 2 and for the at least one micro-optical element 22 to steer the light from the optical fiber 12 through the window 39 into a direction 6-1 and/or spatial region 7-1 perpendicular to the window 39 and through the distal end of the surgical tool 2 through a cutout into the axial direction 6-2 and/or into an axial spatial region 7-2 (into the distal direction of the surgical tool 2) and to capture light from these directions 6-x and/or spatial regions 7-x.

### LIST OF REFERENCE SIGNS

1 Surgical operating system
2 Surgical tool
3 Optical sensor
4 OCT measuring device
5 Distal end
6-x Direction
7-x Spatial region
8-x Distance
9 Vitrectome
10 Vitrectome controller
11 Coherent light source
12 Connector
13 Optical fiber
14 Beam splitter
15 Reference arm
16 Measuring arm
17 Mirror
18 Detector
19 Signal processing device
20 Display device
21 Signal transmitter
22 Micro-optical element
22s Switchable micro-optical element
23 Fiber coupler
24 Feedback
25, 25-x Specified minimum distance
26 Warning message
27 Warning signal
28 Control signal
29 Signal transmitter
30 Robotic system
31 Optical element
32-x E-field vector
33 Further optical element
34-x Light source
35 Depth
36 Depth map
38 Movable element
39 Window
40 Movable blade
41-x Distance between pulse and parasitic pulse
42-x Signal
43-x Parasitic signal
44 Movement direction
50 Tissue
60 Eye
I Intensity
R1 to R4 Reflection surfaces
t1, t2, t3, t4 Times
WD1 OCT working distance
WD2 OCT working distance
λ1, λ2, λ3, λ4 Wavelengths

1. A surgical operating system, comprising:
a surgical tool,
an optical sensor arranged on or in the surgical tool and configured to measure a distance of the surgical tool from a tissue for a plurality of directions and/or for a plurality of mutually separated punctiform spatial regions,
an optical coherence tomography measuring device connected to the optical sensor and configured to determine the respective distance from the tissue for the plurality of directions and/or punctiform spatial regions by means of the sensor and to provide the determined distances.

2. The surgical operating system as claimed in claim 1, wherein the optical sensor comprises an optical fiber which is arranged on or in the surgical tool and at least one micro-optical element which is arranged and/or formed on the surgical tool on and/or in the fiber, with the at least one micro-optical element being configured to steer light from the optical fiber into the plurality of directions and/or into the plurality of mutually separated punctiform spatial regions and input couple light arriving from the plurality of directions and/or the spatial regions into the fiber and with the optical coherence tomography measuring device being connected to the optical fiber.

3. The surgical operating system as claimed in claim 1, wherein the surgical operating system is configured to create and output feedback for a user using at least the determined distance with the smallest value as a starting point.

4. The surgical operating system as claimed in claim 1, wherein the surgical operating system is configured to compare the determined distances with at least one specified minimum distance and create and output
at least one warning message and/or at least one warning signal and/or at least one control signal should the at least one specified minimum distance be undershot.

5. The surgical operating system as claimed in claim 1, wherein the surgical operating system is configured to allow the specification of respective minimum distances for at least some of the plurality of directions and/or spatial regions, to compare the respective distances with the respective specified minimum distances, and to create and output at least one warning message and/or at least one warning signal and/or at least one control signal should the respective specified minimum distance be undershot.

6. The surgical operating system as claimed in claim 1, wherein the surgical tool is an ophthalmological surgical tool.

7. The surgical operating system as claimed in claim 1, wherein the surgical operating system is configured to measure and/or determine at least some of the respective distances simultaneously.

8. The surgical operating system as claimed in claim 1, wherein the optical sensor is configured to guide light of different polarization directions into different directions and/or spatial regions of the plurality of directions and/or spatial regions and capture light arriving therefrom while creating different polarization directions for the different directions and/or spatial regions of the plurality of directions and/or spatial regions, with the optical coherence tomography measuring device being configured to assign the different directions and/or spatial regions on the basis of the polarization direction.

9. The surgical operating system as claimed in claim 1, wherein the optical sensor is configured to guide light from different
wavelength ranges into different directions and/or spatial regions of the plurality of directions and/or spatial regions and to capture light arriving therefrom while maintaining the different wavelength ranges for the different directions and/or spatial regions of the plurality of directions and/or spatial regions, with the optical coherence tomography measuring device being configured to assign the different directions and/or spatial regions on the basis of the wavelength ranges.

10. The surgical operating system as claimed in claim 1, wherein the optical sensor is configured to provide respective light for the different directions and/or spatial regions of the plurality of directions and/or spatial regions with different OCT working distances, with the optical coherence tomography measuring device being configured to assign the different directions and/or spatial regions on the basis of the OCT working distances.

11. The surgical operating system as claimed in claim 1, wherein the optical sensor is configured to guide light of different intensities into different directions and/or spatial regions of the plurality of directions and/or spatial regions and capture light arriving therefrom, with the optical coherence tomography measuring device being configured to assign the different directions and/or spatial regions on the basis of the intensities.

12. The surgical operating system as claimed in claim 1, wherein the optical sensor is configured to guide light of different spectral widths into different directions and/or spatial regions of the plurality of directions and/or spatial regions and capture light arriving therefrom, with the optical coherence tomography measuring device being configured to assign the different directions and/or spatial regions on the basis of the different spectral widths.

13. The surgical operating system as claimed in claim 2, wherein the optical fiber is a multi-mode fiber, with the at least one micro-optical element being configured to guide light of different modes from the optical fiber into different directions and/or spatial regions of the plurality of directions and/or spatial regions and input couple light arriving therefrom back into the respective original mode of the optical fiber, with the optical coherence tomography measuring device being configured to assign the different directions and/or spatial regions on the basis of the modes.

14. The surgical operating system as claimed in claim 2, wherein the optical fiber is a multi-core fiber, with the at least one micro-optical element being configured to guide light from different cores from the optical fiber into different directions and/or spatial regions of the plurality of directions and/or spatial regions and input couple light arriving therefrom back into the respective original cores of the optical fiber, with the optical coherence tomography measuring device being configured to assign the different directions and/or spatial regions on the basis of the cores.

15. The surgical operating system as claimed in claim 1, wherein the surgical operating system is configured to temporally modulate light guided in different directions and/or spatial regions of the plurality of directions and/or spatial regions by means of the optical sensor, with the optical coherence tomography measuring device being configured to assign the different directions and/or spatial regions on the basis of the temporal modulation.

16. The surgical operating system as claimed in claim 1, wherein at least some of the respective distances are measured and/or determined sequentially.

17. The surgical operating system as claimed in claim 16, wherein the surgical operating system is configured to temporally sweep a wavelength of the light guided into the different directions and/or spatial regions, with the optical sensor being configured to successively guide the light, matched to the wavelength, into the different directions and/or spatial regions and to capture light arriving therefrom, with the optical coherence tomography measuring device being configured to assign the different directions and/or spatial regions in a manner matched to the wavelength.

18. The surgical operating system as claimed in claim 17, wherein the optical sensor comprises at least one dichroic mirror as micro-optical element.

19. The surgical operating system as claimed in claim 16, wherein the optical sensor comprises a switchable micro-optical element as micro-optical element, in which it is possible to set the direction and/or the spatial region into which the light is guided, with the surgical operating system being configured to control the switchable micro-optical element in such a way that the light is successively guided into the different directions and/or spatial regions and is captured therefrom again, with the optical coherence tomography measuring device being configured to assign the different directions and/or spatial regions in a manner matched to the control of the switchable micro-optical element.

20. The surgical operating system as claimed in claim 15, wherein the surgical tool comprises a movable element, with the movable element being configured to guide light into the different directions and/or spatial regions by means of a movement of the movable element and to capture the said light from these directions and/or spatial regions, with the optical coherence tomography measuring device being configured to assign the different directions and/or spatial regions in a manner matched to the movement of the movable element.

21. The surgical operating system as claimed in claim 1, wherein the optical coherence tomography measuring device is configured to take account of a position, an orientation and/or a movement of the surgical tool when determining the distances.

22. The surgical operating system as claimed in claim 1, wherein the optical coherence tomography measuring device is configured to take account of a model of an organ or body part when determining the distances.

23. The surgical operating system as claimed in claim 1, wherein the optical coherence tomography measuring device is configured to take account of at least one calibration curve when determining the distances.

24. The surgical operating system as claimed in claim 1, wherein the optical sensor is configured so that at least one spectral property of light guided into different directions and/or spatial regions differs, with the optical coherence tomography measuring device being configured to assign the different directions and/or spatial regions while taking account of the respective spectral properties.

25. The surgical operating system as claimed in claim 24, wherein the spectral properties comprise at least one of the following: a signal width, a distance between a signal and a parasitic signal, a property of a parasitic signal.

26. A surgical tool, comprising:
an optical sensor arranged on or in the surgical tool and configured to measure a distance of the surgical tool from a tissue for a plurality of directions and/or for a plurality of mutually separated punctiform spatial regions.

27. The surgical tool as claimed in claim 26, wherein the sensor comprises an optical fiber which is arranged on or in the surgical tool and at least one micro-optical element which is arranged and/or formed on the surgical tool on
and/or in the fiber, with the at least one micro-optical element being configured to steer light from the optical fiber into a plurality of directions and/or into a plurality of mutually separated punctiform spatial regions and input couple light arriving from the plurality of directions and/or the spatial regions into the fiber.

28. A method for safeguarding a surgical operation,
wherein a distance of a surgical tool from a tissue is measured for a plurality of directions and/or for a plurality of mutually separated punctiform spatial regions by means of an optical sensor arranged on or in the surgical tool,
wherein an optical coherence tomography measuring device connected to the optical sensor is used to determine a respective distance from the tissue for the plurality of directions and/or spatial regions using a sensor signal from the optical sensor as a starting point, and the determined distances are provided.
